(19) United States
(12) Patent Application Publication
(10) Pub. No.: US 2012/0294504 A1
(43) Pub. Date: Nov. 22, 2012

(54) METHOD FOR PROVIDING AN IMAGE DATA RECORD WITH SUPPRESSED ALIASING ARTIFACTS OVERLAPPING THE FIELD OF VIEW AND X-RAY IMAGE RECORDING APPARATUS

(75) Inventor: Yiannis Kyriakou, Spardorf (DE)

(73) Assignee: SIEMENS AKTIENGESELLSCHAFT, Munich (DE)

(21) Appl. No.: 13/471,978

(22) Filed: May 15, 2012

(30) Foreign Application Priority Data
May 16, 2011 (DE) 102011075904.2

(51) Int. Cl. G06K 9/40; A61B 6/03

(52) U.S. Cl. 382/132; 378/19

(57) ABSTRACT

A method is disclosed. In at least one embodiment, the method includes obtaining an x-ray image data record with respect to the biological object by way of the x-ray image recording apparatus; obtaining a comparison image data record with respect to the biological object relating to a three-dimensional surface structure of the biological object; assigning data of the comparison image data record to data of the x-ray image data record by determining a predeterminable geometric assignment rule; and extending and/or amending data of the x-ray image data record as a function of data of the comparison image data record for at least one part of such data of the x-ray image data record, which can be assigned to an aliasing artifact overlapping the field of view.

### PRIORITY STATEMENT

The present application hereby claims priority under 35 U.S.C. §119 to German patent application number DE 10 2011 075 904.2 filed May 16, 2011, the entire contents of which are hereby incorporated herein by reference.

### FIELD

At least one embodiment of the invention generally relates to a method for providing an image data record relating to a biological object with suppressed aliasing artifacts overlapping the field of view, which are caused by an incomplete geometric capture of the biological object by way of an x-ray image recording apparatus. At least one embodiment of the invention also generally relates to an x-ray image recording apparatus having an image evaluation apparatus which is embodied to implement such a method.

### BACKGROUND

The x-ray and/or tomography images obtained by x-ray image recording apparatuses, in particular computed tomography system, may comprise various image artifacts. One type of image artifact is to be attributed to the incomplete capture of the measured object during the measuring process in terms of its geometric extension. One part of the measuring object is outside of the field of view and is, in this way, truncated in terms of the image obtained therefrom. The image artifacts resulting herefrom are referred to below as aliasing artifacts overlapping the field of view. They have an essential role particularly in computed tomography systems since a three-dimensional image obtained by way of back projection is frequently based on a plurality of projection images which do not always capture the object to be measured in its entirety or completely. The object is namely not constantly completely inside the field of view during the measuring process.

This unwanted data reduction may be significant with all computed tomographical scan apparatuses, but nevertheless plays an important role particularly with flat panel computed tomographs (see “W. A. Kalender and Y. Kyriaku. Flat-detector CT. Eur Radiol. (11):2767-79,2007”). With flat panel detector computed tomographs, the field of view of the detector which can be captured during the measurement only amounts to approximately 20-25 cm in diameter. This restriction renders the prevention of aliasing artifacts overlapping the field of view almost impossible. Aliasing artifacts overlapping the field of view impair the quality of a resulting x-ray and/or tomography image. In this way the artifacts not only appear in the vicinity of the image edge, but also influence central regions of the recorded image.

Aliasing artifacts overlapping the field of view would then not appear for instance if the x-ray radiation was not attenuated at all border areas of the field of view. A defined transition with respect of the absorption values to zero would then result. If this transition does however not occur correctly, with computed tomography recordings in particular according to the filtered back projection (see for instance “A. C. Kak and M. Slaney. Principles of Computerized Tomographic Imaging. IEEE Press, 1988”, “L. A. Feldkamp, L. C. Davis, and J. W. Kress. Practical cone-beam algorithm. J. Opt. Soc. Am. A, 1(6):612-619, 1984”) the effect that aliasing artifacts overlapping the field of view occur and an apparent increase in the x-ray radiation attenuation values to the image edges is observed. A pale white ring is produced beyond the edge of the field of view in the computed tomography image. Strip-like artifacts also result outside of the actual field of view range.

Aliasing artifacts overlapping the field of view are generally suppressed such that image areas at the edge of the field of view, to which attenuation values greater than zero are assigned, are extrapolated such that a smooth value response to the x-ray absorption value of zero is produced. According to a known method, the truncated areas in the computed tomography projection images used for the back projection are extrapolated toward an attenuation value of zero and it is only then that the filtered back projection is implemented. Within the scope of this extrapolation method, objects are approached for instance by way of a water cylinder (see “Hsieh J, Chao E, Thibault J, Grekowicz B, Horst A, McOlash S and Myers T J, 2004, A novel reconstruction algorithm to extend the CT scan field-of-view Med. Phys. 31, 2385-91”). The patient as a whole can also be approximated as a water ellipsoid, so that in this way data is available for the extrapolation (see “Maltz J S, Bose S, Shukla H P and Bani-Hashemi A R, 2007, CT truncation artifact removal using water-equivalent thicknesses derived from truncated projection data Proc. IEEE Eng. Med. Biol., Soc. 2007. 2907-11”).

A quadratic extrapolation is known for instance from “Sourbelle K, Kachelrieg M and Kalender W A, 2005, Reconstruction from truncated projections in CT using adaptive detruncation Eur. Radial. 15, 1008-14”, when a so-called sinogram interpolation is described in “Zamyatin A A and Nakanishi S, 2007, Extension of the reconstruction field of view and truncation correction using sinogram decomposition Med. Phys. 34, 1593-60”. Further extrapolation methods are known from the following publications: “Janoop K P and Rajgopal K, 2007, Estimation of missing data using windowed linear prediction in laterally truncated projections in cone-beam CT Proc. IEEE Eng. Med. Biol. Soc. 2007, 2903-6”, “Starman J, Pelc N, Strobe N and Fahrig R, 2005, Estimating 0th and 1st moments in C-arm CT data for extrapolating truncated projections Proc. SPIE 5747, 378-87” and “Sourbelle K, KachelrieB M and Kalender W A, 2005, Reconstruction from truncated projections in CT using adaptive detruncation Eur. Radiol. 15, 1008-14”.

### SUMMARY

The methods known from the prior art have the objective of improving the image quality within the field of view range, but nevertheless impair an image modification and/or quality improvement outside of the field of view measurement. In the event that several border areas are truncated in the computed tomography projection images, additional serious disadvantages result. With the majority of methods, at least one non-truncated projection image is needed in order to ensure fulfillment of the consistency criterion. A conversion of 3D into 2D data is frequently extremely time-consuming. Very reduced data records, which are the rule in the case of flat panel detector computer tomographs, cannot be overcome by the conventional methods with respect to aliasing artifacts overlapping the field of view. In addition, anatomical information is frequently lost. The contour of a patient is generally not reproduced correctly, which hampers a treating physician during an operation for instance, in terms of navigating instruments in the body of the patient with the aid of the computed tomography image.

At least one embodiment of the invention provides a method and an x-ray image recording apparatus with which aliasing artifacts overlapping the field of view can be suppressed even better.

A method and an x-ray image recording apparatus are disclosed.

The inventive method of at least one embodiment is used to provide an image data record of a biological object with suppressed aliasing artifacts overlapping the field of view, which are caused by an incomplete geometric capture of the biological object by way of an x-ray image recording apparatus. The method includes:

a) obtaining an x-ray image data record with respect to the biological object by way of the x-ray image recording apparatus;

b) obtaining a comparison image data record with respect to the biological object relating to a three-dimensional surface structure of the biological object;

c) assigning data of the comparison image data record to data of the x-ray image data record by determining a predeterminable geometric assignment rule; and

d) extending and/or amending data of the x-ray image data record as a function of data of the comparison image data record for at least one part of such data of the x-ray image data record, which can be assigned to an aliasing artifact overlapping the field of view.

An inventive x-ray image recording apparatus of at least one embodiment includes an x-ray source, a detector and an image evaluation apparatus, wherein the image evaluation apparatus is embodied to execute at least one embodiment of the inventive method. The x-ray image recording apparatus may be embodied in particular as a computed tomograph.

### BRIEF DESCRIPTION OF THE DRAWINGS

The invention is described in more detail below with the aid of example embodiments, in which:

FIG. 1 shows a schematic representation of a computed tomograph according to the prior art;

FIG. 2A shows a schematic representation of a computed tomography having an x-ray source and a TOF camera on a shared side of an x-ray C-arm;

FIG. 2B shows a schematic representation of a computed tomograph having an x-ray source and a TOF camera on opposite sides of an x-ray C-arm;

FIG. 3 shows a schematic illustration of an exemplary embodiment of the inventive method; and

FIG. 4 shows a flow chart of an example embodiment of the inventive method.

Identical or functionally identical elements are provided with the same reference characters in the figures.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be rearranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The inventive method of at least one embodiment is used to provide an image data record of a biological object with suppressed aliasing artifacts overlapping the field of view, which are caused by an incomplete geometric capture of the biological object by way of an x-ray image recording apparatus. The method includes:

a) obtaining an x-ray image data record with respect to the biological object by way of the x-ray image recording apparatus;

b) obtaining a comparison image data record with respect to the biological object relating to a three-dimensional surface structure of the biological object;

c) assigning data of the comparison image data record to data of the x-ray image data record by determining a predeterminable geometric assignment rule; and

d) extending and/or amending data of the x-ray image data record as a function of data of the comparison image data record for at least one part of such data of the x-ray image data record, which can be assigned to an aliasing artifact overlapping the field of view.

The x-ray image data record can be extended and/or amended by way of the comparison image data record such that direct consideration can be given here to the shape and form of the biological object (e.g. the anatomical conditions of a person). Transitions in otherwise truncated areas in the image can be reconstructed very precisely in this way. Aliasing artifacts overlapping the field of view can be reduced particularly effectively in this way. Even if only a small subarea of the object is captured in the x-ray image data record, the areas which are not captured can be very effectively reconstructed with the aid of the comparison image data record. The method is thus also suited to the instance of pronounced aliasing artifacts overlapping the field of view. Within the scope of the method, the shape and size of the object are not only considered approximately but instead reproduced particularly accurately and realistically on the basis of the comparison image data record. A particularly precise extension and/or amendment of data of the x-ray image data record is possible in this way.

It is possible to provide the geometric assignment rule by way of matching the coordinate system assigned to the x-ray image data record in the step of assigning the data of the comparison image data record to data from the x-ray image data, record. The determination of the assignment rule nevertheless preferably takes placed in step c) by way of registering the x-ray image data record with the comparison image data record. Registering is understood to mean in particular the image registration by way of positionally and dimensionally-correct assignment of the respective image data record. An imaging rule can in particular be specified, by which the x-ray and comparison image data record are linked with one another with respect to the image and/or data similarity. This embodiment allows for the assignment in step c) to also be fully automatic and uncomplicated without previous knowledge of the association of absolute coordinates of the x-ray and comparison image data record.

In at least one embodiment, a 3D image data record is obtained in step a) and a 3D comparison image data record is obtained in step b). The 3D x-ray image data record may in particular be obtained such that several 2D projection x-ray images are initially obtained and then the 3D x-ray image data record is generated by way of back projection from these 2D x-ray image data records. The 3D comparison image data record may in particular be obtained such that the object is captured at different angles using measuring technology and a 3D surface image, which corresponds to the 3D comparison image data record, is generated. The actual form and/or shape of the biological object can in this way be particularly precisely captured both in the x-ray and also in the comparison image data record, thereby facilitating the data assignment in step c). Comparison images precisely describing the shape of the biological object are provided, by which the x-ray image data can be extended or amended such that the conditions actually existing are reproduced particularly well.

Alternatively, a 2D x-ray image data record is obtained in step a), and a 2D comparison image data record is obtained in step b). The 2D image data record may in particular be an x-ray projection image relating to a data record, whereas the 2D comparison image data record can be obtained in particular by determining a density allocation of the biological object which can be derived from the three-dimensional surface structure of the biological object. An item of depth information is then available by way of the 2D comparison image data record. Provision can then in particular be made for an image registration to take place with the aid of the x-ray radiation attenuation (gray-scale value) in the x-ray projection image and the depth information in the 2D comparison image. The assignment of the data of the comparison image data record to the data of the x-ray image data record turns out to be particularly simple by comparing intensity values.

The extension and/or amendment of the data of the x-ray image data record by way of a smoothing extrapolation method, in particular a quadratic extrapolation, of data of the x-ray image record, which can be assigned to an border area of the geometric capture of the biological object on account of a restricted field of view of the x-ray image recording apparatus, preferably takes place in step b). Provision can in particular be made for border areas truncated in the x-ray image to be extrapolated on the basis of the comparison image data. The assumption of a simplified model is not necessary for the extrapolation. Instead, the shape, form and geometry of the biological object can be taken directly into account within the scope of the extrapolation by way of the comparison image data record. The exact contour of the biological object can thus be taken into account during the extrapolation. The resulting x-ray images are particularly meaningful since they reproduce the reality very effectively.

The comparison image data record or a data record from which the comparison image data record is derived, is preferably obtained in step b) by way of a transit time technique. Within the scope of the transit time technique, provision can in particular be made for a distance measurement to take place by way of transit time measurement of an electro-magnetic wave or sound wave and thus the surface structure of the biological object can be reconstructed from the distances. A measurement of the three-dimensional surface structure of the biological object is then particularly simple. Additional marker or measuring elements on the object itself are not needed.

An embodiment of the method includes a calibration step, in which a clear assignment between a first coordinate system, which is linked to the x-ray image data record, and a second coordinate system, which is linked to the comparison image data record, takes place. A clear assignment between the coordinate systems is then possible without any problem and the x-ray image and comparison image data record can then be directly related to one another.

Step c), in at least one embodiment, includes the following sub steps:

c1) determining data in the x-ray image data record, which can be assigned to a predeterminable first subarea of the biological object;

c2) determining data in the comparison image data record, which can be assigned to a second subarea of the biological object, which at least partially includes the predetermined first subarea;

c3) selecting such data from the data determined in step c2) which can be assigned to the first subarea.

The data selected in step c3) therefore extend in particular the incomplete data record in step c1) by way of the data of the comparison image data record.

Step d) then, in at least one embodiment, includes the following sub step:

d1) extending and/or amending data from data specified in step c1) as a function of the data selected in step c3.

Truncated areas in the x-ray image can then be extended in particular on the basis of comparison data, which correspond to this truncated image area, by way of extrapolation. A modified x-ray image data record then results in particular, which includes simulated x-ray data in image areas which actually appear exclusively in the comparison image.

A field of view of the x-ray image recording apparatus is preferably completely included in a field of view for obtaining the comparison image data record. Provision may in particular be made for the field of view of a measuring apparatus for obtaining the comparison image data record to completely include the field of view of the x-ray image recording apparatus. An extension of the x-ray image data record is then possible in the entire border area.

An inventive x-ray image recording apparatus of at least one embodiment includes an x-ray source, a detector and an image evaluation apparatus, wherein the image evaluation apparatus is embodied to execute at least one embodiment of the inventive method. The x-ray image recording apparatus may be embodied in particular as a computed tomograph.

The x-ray image recording apparatus of at least one embodiment includes a sensor, which is embodied so that the comparison image data record or a data record, from which the comparison image data record can be derived, is captured by way of a transmit time measurement. The sensor may then be in particular a TOF (Time of Flight) camera. Three-dimensional surface structures can in this way be captured in a particularly precise and simple fashion. An integration of such a sensor in existing computed tomographs is simple.

The x-ray source and the sensor, in at least one embodiment, are fastened to a shared holding apparatus, in particular an x-ray C-arm. A shared field of view of the x-ray source and sensor is then particularly easy to realize. The x-ray source and the sensor are preferably positioned on a shared side facing the biological object. The same angular views of the object can then be captured by the x-ray source and the sensor at the same point in time. It is then particularly easy to relate the x-ray image and comparison image spatially to one another.

Provision can however also preferably be made for the sensor to be positioned directly on or adjacent to the detector.

The sensor can preferably be embodied to implement a transit time measurement by way of non-visible light, in particular infrared light. Parasitic inductions by ambient light can then be ruled out particularly effectively.

The preferred embodiments and their advantages which are shown with respect to the inventive method apply accordingly to the inventive x-ray image recording apparatus.

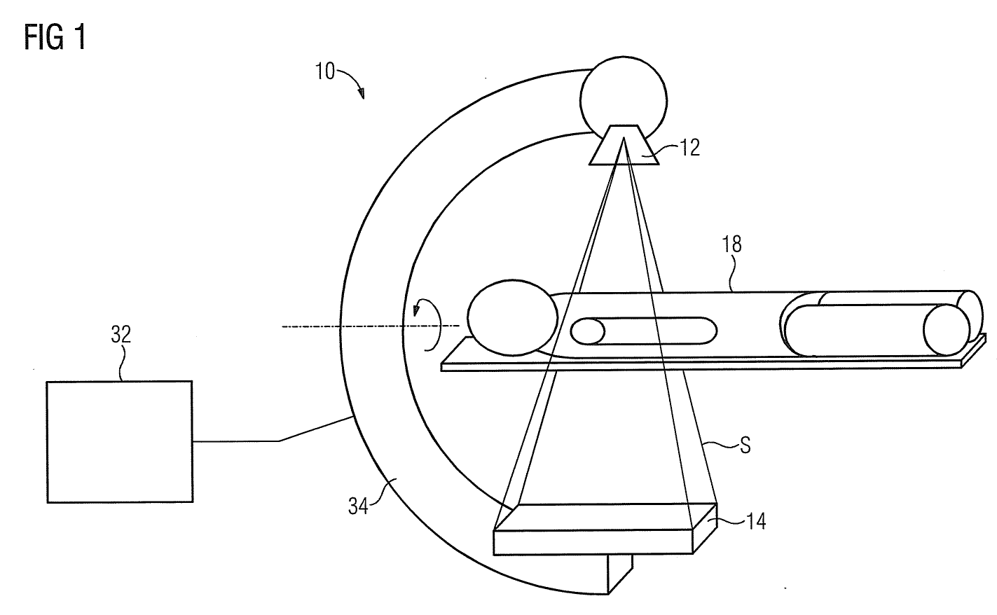

FIG. 1 shows a computed tomograph system 10 having an x-ray C-arm 24, to one end of which an x-ray source 12 is fastened and emits x-ray radiation S in the direction of an x-ray detector 14. A patient 18 is arranged on a couch between the x-ray source 12 and the x-ray detector 14, wherein a subarea and/or body part of the patient 18 is irradiated by the x-ray radiation S.

The x-ray C-arm 26 is embodied to be rotary and can in this way capture the patient 18 from different perspectives and/or at different angles. In this way, different x-ray projection images can be captured by way of the x-ray detector 14, which are transmitted to a computer 32. A 3D image data record can be reconstructed in the computer 32 from the projection images by way of a back projection method.

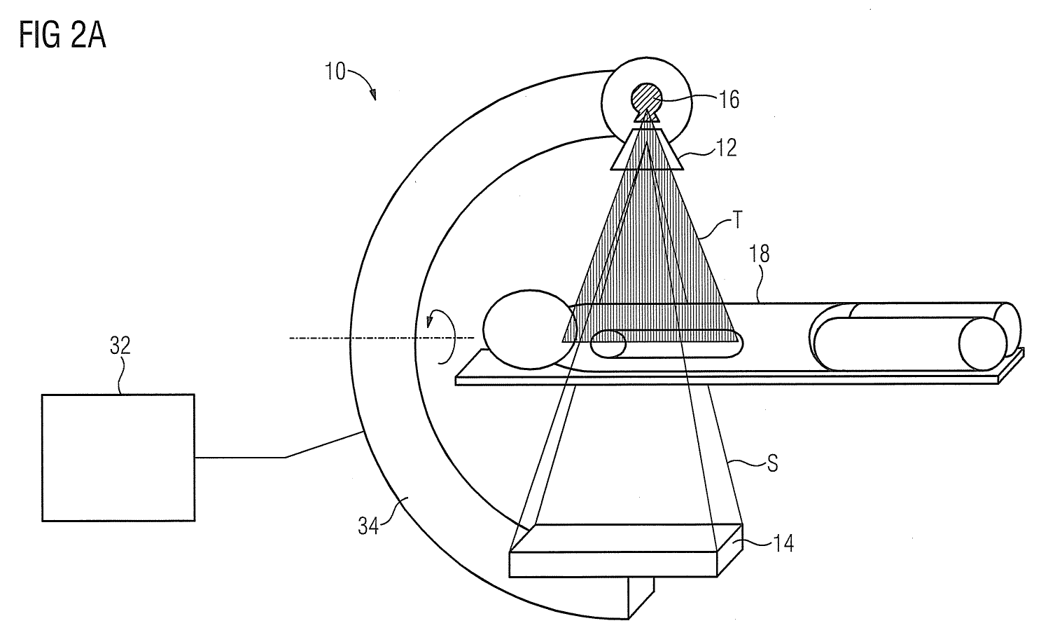

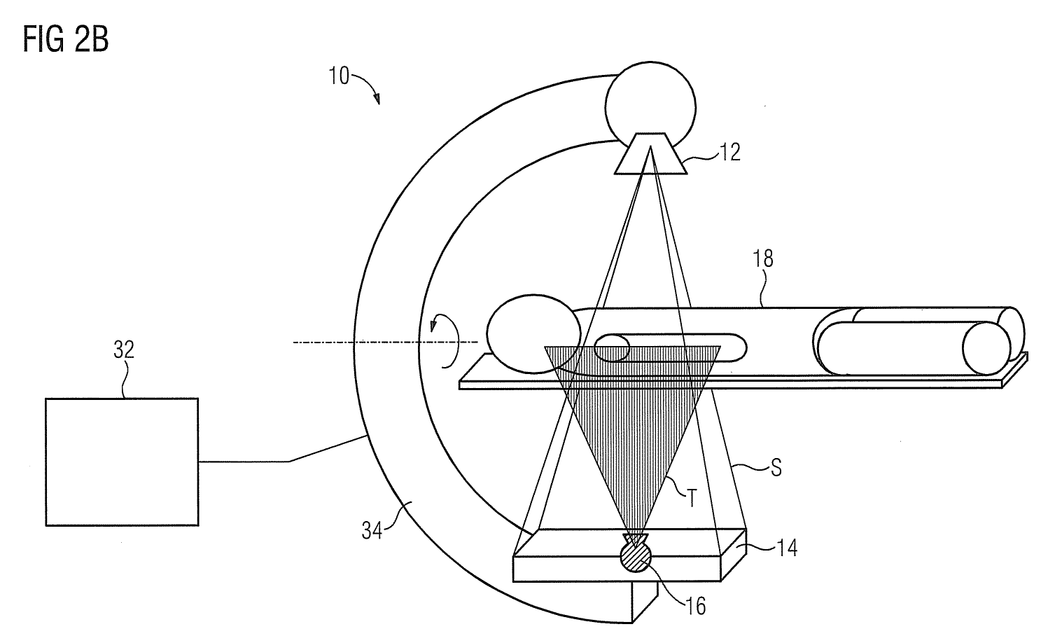

According to FIG. 2A and 2B, from now on a TOF (Time of Flight) camera 16 is attached to the x-ray C-arm 34 in addition to the x-ray source 12 and the x-ray detector 14. The TOF capture area T of the TOF camera 16 completely covers the capture and/or irradiation area of the x-ray radiation. S. In this way the x-ray source 12 and TOF camera 16, as shown in FIG. 2A, can be mutually positioned on the x-ray C-arm 34 on a side facing the biological object. Alternatively, it is however also possible, as shown in FIG. 2B, for the TOF camera 16 to be positioned directly on the detector 14, whereby the TOF camera 16 and the x-ray source 12 are then positioned on different sides of the biological object.

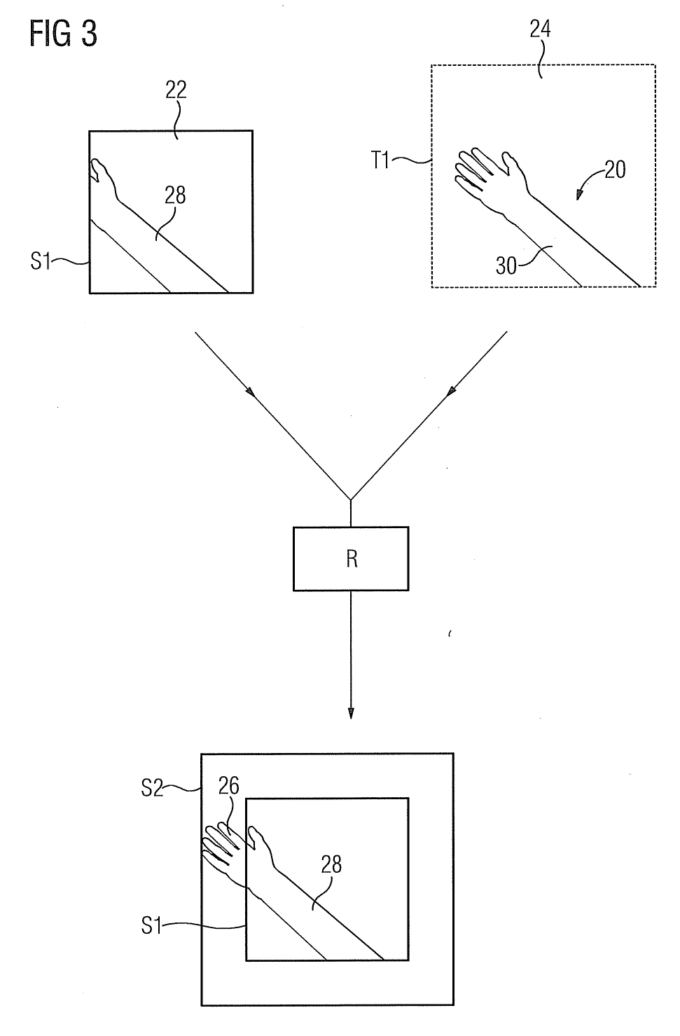

FIG. 3 schematically reproduces the image comparison and processing method running in the computer 32. In the exemplary embodiment, an arm 20 of the patient 18 is observed. The arm 20 is captured in a transit time method by way of the TOF camera 16 and is represented in the form of a TOF image 24. Aside from the contour of the arm 20, the TOF image 24 also contains an item of depth information relating to the three-dimensional surface structure of the arm 20. This depth information allows conclusions to be drawn as to the height and/or density of the biological material present per pixel in the TOF image 24. A three-dimensional surface model of the arm 20 can be calculated in the computer 32. The typical image recording rate with the TOF camera 15 amounts to 100 images per second. Depending on the construction type, the TOF camera 16 exhibits a specific TOF field of view T1, so that a specific subarea 30 of the arm 20 is captured.

FIG. 3 also shows an x-ray image 22 (in this case an x-ray projection image), which was obtained for a specific angular position of the x-ray C-arm 34 by way of the x-ray source 12 and x-ray detector 14. The x-ray field of view S1, which corresponds to the field of view of the x-ray source 12 and x-ray detector 14, is smaller than the TOF field of view T1, so that a subarea 28 of the arm 20 is captured, which is smaller than the subarea 30 in the TOF image 24.

In order to produce a relationship between the TOF image 24 and the x-ray image 22, it is necessary to geometrically relate the coordinate systems of the x-ray arrangement (x-ray source 12 and x-ray detector 14) and the TOF camera 16 to one another. In the exemplary embodiment, this takes place in step R by a method for image registration. The computer 32 implements a comparison algorithm, in order to produce as good an image overlap of the x-ray image 22 and TOF image 24 as possible. The necessary coordinate transformation can take place in this way. Additional image manipulation steps (e.g. smoothing) can be provided.

As apparent from the x-ray image 22, a hand of the patient 18 is partially truncated at the arm 20 in the image. This may result in aliasing artifacts overlapping the field of view in resulting computed tomography images (e.g. by back projection of the x-ray image 22, embodying a 3D image data record and subsequently forward projection). With the aid of the TOF image 24, correction of the aliasing artifacts suppressing the field of view is now provided. A smoothing extrapolation is herewith performed on the x-ray image 22. Image data of the subarea 30 in the TOF image 24, to which no image data of the subarea 28 in the x-ray image 22 corresponds (in other words image data of the TOF image 24, which belongs to an extension area 26), is used here as a basis for the extrapolation.

Extrapolation methods known from the prior art, like for instance the quadratic extrapolation, can be used here. In order to be able to implement the extrapolation, no more assumptions need be made about the truncated area (in other words about the extension area 26). The form and/or shape and/or contour and/or density of the arm 20 in the extension area 26 are namely known from the TOF image 24. The x-ray field of view S1 can in this way be extended to an apparently and/or virtually enlarged x-ray field of view in the form of the overall field image field S2.

The TOF camera 16 is advantageous in that with its three-dimensional model of the arm 20, images can be obtained in real time. Instead of a TOF camera 16, several TOF sensors can also be used, which can also be positioned outside of the x-ray C-arm 24 in the room. These TOF sensors need not necessarily be fastened to the x-ray C-arm 24. Additional sensor elements, which have to be attached to the patient 18, are not needed. A synchronized data recording between the x-ray measuring apparatus and the TOF camera 16 is also not necessarily essential.

The method is suited not only to conventional computed tomography systems but can also be used in conjunction with flat panel computed tomography systems, multislice CT or PET/CT scanners.

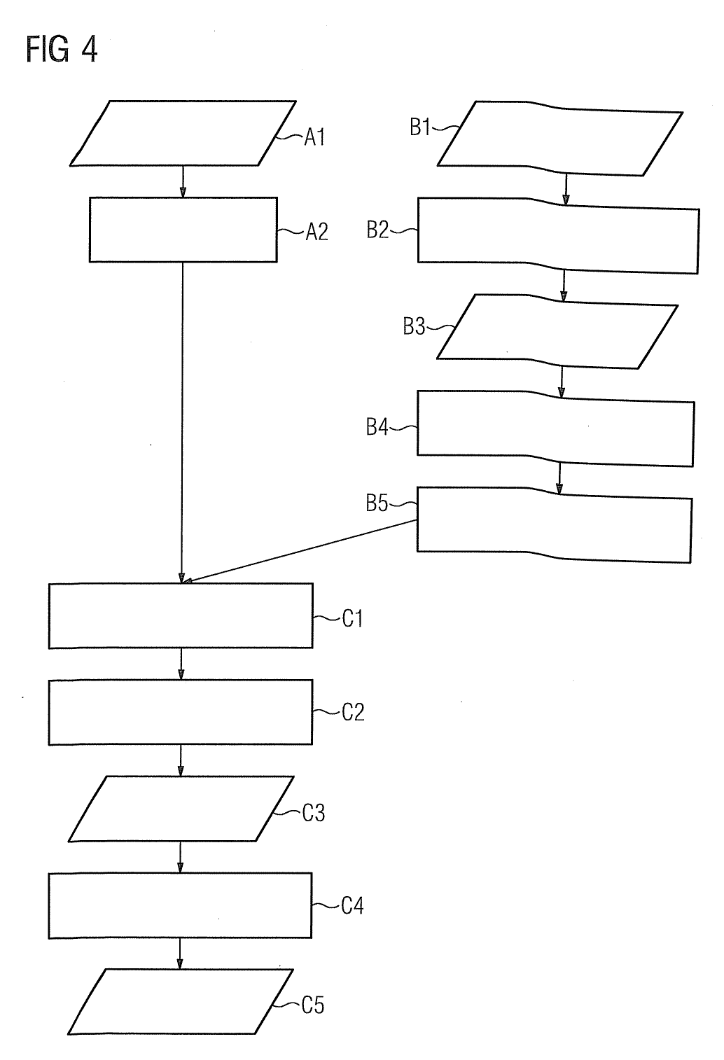

The method is briefly mentioned again with the aid of FIG. 4. In a step A1, an x-ray projection image is provided, in which an object is not completely captured, so that with a further processing of this image, aliasing artifacts overlapping the field of view may result. In a step A2, the associated object limits are determined. In a step B1, measuring data in the form of a so-called scatter plot are determined with the TOF camera 16. In a step B2, an interpolation of this measuring data takes place so that smooth surfaces are generated in the image. A surface model is herewith produced in step B3. This is now geometrically related to the x-ray projection image in step B4 by way of a coordinate transformation. A perspective transformation also takes place in a step B5. In a step C1, the thus resulting image data records are compared and the contour of the object in the x-ray projection image is adjusted. Step C3 combines image manipulation steps by way of extrapolation and smoothing. The result in step C3 is the provision of an x-ray projection image, the image areas of which were previously truncated are now extended. In a step C4, a filtered back projection can now take place so that a corrected 3D image data record is available in step C5, in which aliasing artifacts overlapping the field of view are suppressed.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for providing an image data record relating to a biological object with suppressed aliasing artifacts overlapping a field of view, caused by an incomplete geometric capture of the biological object by way of an x-ray image recording apparatus, the method comprising:
a) obtaining an x-ray image data record with respect to the biological object by way of the x-ray image recording apparatus;
b) obtaining a comparison image data record with respect to the biological object relating to a three-dimensional surface structure of the biological object;
c) assigning data of the comparison image data record to data of the x-ray image data record by determining a geometric assignment rule; and
d) at least one of extending and amending data of the x-ray image data record as a function of data of the comparison image data record for at least one part of the data of the x-ray image data record, assignable to an aliasing artifact overlapping the field of view.

2. The method as claimed in claim 1, wherein, in step c), the determination of the assignment rule takes place by way of registering the x-ray image data record with the comparison image data record.

3. The method as claimed in claim 1, wherein a 3D x-ray image data record is obtained in step a) and a 3D comparison image data record is obtained in step b).

4. The method as claimed in claim 1, wherein, in step a), a 2D x-ray image data record is obtained and in step b), a 2D comparison image data record is obtained.

5. The method as claimed in claim 1, wherein, in step d), the at least one of extension and amendment of the data of the x-ray image data record takes place by way of a smoothing extrapolation method, in particular a quadratic extrapolation, of data of the x-ray image data record, which is assignable to a border area of the geometric capture of the biological object on account of a restricted field of view of the x-ray image recording apparatus.

6. The method as claimed in claim 1, wherein, in step b), the comparison image data record or a data record from which the comparison image data record is derived, is obtained by way of a transit time method.

7. The method as claimed in claim 1, further comprising:
calibrating, such that a clear assignment, between a first coordinate system linked to the x-ray image data record and a second coordinate system linked to the comparison image data record, takes place.

8. The method as claimed in claim 1, wherein step c) includes:
c1) determining data in the x-ray image data record, assignable to a first subarea of the biological object,
c2) determining data in the comparison image data record, assignable to a second sub area of the biological object, which at least partially includes the first sub area, and
c3) selecting data from the data determined from step c2), which cannot be assigned to the first subarea; and wherein step d) includes:
d1) at least one of extending and amending data from the data determined in step cl as a function of the data selected in step c3).

9. The method as claimed in claim 1, wherein a field of view of the x-ray image recording apparatus is completely included in the field of view for obtaining the comparison image data record.

10. An x-ray image recording apparatus, comprising:
an x-ray source;
a detector; and
an image evaluation apparatus, configured to
obtain an x-ray image data record with respect to the biological object by way of the x-ray image recording apparatus,
obtain a comparison image data record with respect to the biological object relating to a three-dimensional surface structure of the biological object,
assign data of the comparison image data record to data of the x-ray image data record by determining a geometric assignment rule, and
at least one of extend and amend data of the x-ray image data record as a function of data of the comparison image data record for at least one part of the data of the x-ray image data record, assignable to an aliasing artifact overlapping the field of view.

11. The x-ray image recording apparatus as claimed in claim 10, further comprising a sensor, configured to capture the comparison image data record or a data record from which the comparison image data record is derivable, by way of a transmit time measurement.

12. The x-ray image recording apparatus as claimed in claim 11, wherein the x-ray source and the sensor are fastened to a shared holding apparatus.

13. The x-ray image recording apparatus as claimed in claim 11, wherein the x-ray source and the sensor are positioned on a shared side facing the biological object.

14. The x-ray image recording apparatus as claimed in claim 11, wherein the sensor is positioned directly on or adjacent to the detector.

15. The x-ray image recording apparatus as claimed in claim 11, wherein the sensor is embodied to implement a transit time measurement by way of non-visible light.

16. The method as claimed in claim 2, wherein a 3D x-ray image data record is obtained in step a) and a 3D comparison image data record is obtained in step b).

17. The method as claimed in claim 4, wherein the 2D x-ray image data record is a data record relating to an x-ray projection image, and wherein, in step b), the 2D comparison image data record is obtained by determining a density allocation of the biological object which is derivable from the three-dimensional surface structure of the biological object.

18. The x-ray image recording apparatus of claim 10, wherein the x-ray recording apparatus is a computed tomograph system.

19. The x-ray image recording apparatus as claimed in claim 10, wherein the x-ray source and the sensor are fastened to a shared holding apparatus.

20. The x-ray image recording apparatus as claimed in claim 12, wherein the shared holding apparatus is an x-ray C-arm.

21. The x-ray image recording apparatus as claimed in claim 19, wherein the shared holding apparatus is an x-ray C-arm.

22. The x-ray image recording apparatus as claimed in claim 12, wherein the x-ray source and the sensor are positioned on a shared side facing the biological object.

23. The x-ray image recording apparatus as claimed in claim 12, wherein the sensor is positioned directly on or adjacent to the detector.

24. The x-ray image recording apparatus as claimed in claim 15, wherein the non-visible light is infrared light.

25. A computer readable medium including program segments for, when executed on a computer device, causing the computer device to implement the method of claim 1.
